(19) United States
(12) Patent Application Publication
(10) Pub. No.: US 2003/0109895 A1
(43) Pub. Date: Jun. 12, 2003

(54) Keyed lancet with generic mounting compatibility

(76) Inventors: William C. Taylor, Alpharetta, GA (US); Richard W. Levaughn, Talking Rock, GA (US); Christopher J. Ruf, Atlanta, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/202,782

(22) Filed: Jul. 24, 2002

Related U.S. Application Data
(60) Provisional application No. 60/338,970, filed on Dec. 7, 2001.

(51) Int. Cl. A61B 17/32

(52) U.S. Cl. 606/181

(57) ABSTRACT

A lancet having a coupling profile that is compatible with lancet holding features of standard lancing devices, but is keyed for mounting in a non-standard lancing device having a cooperating keyed coupling profile; a lancing device having a keyed lancet coupling for receiving a correspondingly keyed lancet but preventing use of unauthorized lancets; and a method of preventing use of unauthorized lancets with a lancing device.

### CROSS-REFERENCE TO RELATED APPLICATIONS

This application is entitled to the benefit of U.S. Provisional Patent Application Serial No. 60/338,970 filed in the United States Patent and Trademark Office on Dec. 7, 2001, which is hereby incorporated herein by reference in its entirety for all purposes.

### FIELD OF THE INVENTION

The present invention relates generally to medical devices, and more particularly to a lancing device and lancets for penetrating the skin of a human or animal subject for sampling of blood and/or other body fluids.

### BACKGROUND OF THE INVENTION

Lancing devices are used to penetrate the skin of a subject and obtain a sample of blood or other body fluid, as in the testing of blood sugar levels by diabetics. Typically, a lancet having a sharp point is mounted to a lancet coupling within a housing portion of a lancing device. The lancet is translationally driven by a spring or other biasing means to cause the sharp point of the lancet to extend a small distance through an opening in the housing and penetrate into the subject's skin, creating a wound from which the sample of body fluid is collected. The used lancet can then be removed from the lancing device and replaced with a new lancet for further sampling, as required. Thus, the lancing device is typically reusable, whereas the lancets are disposable and purchased in quantity.

Manufacturers and sellers of lancets, therefore, often prefer to provide “standard” lancets (i.e., lancets that are compatible with multiple types of existing lancing devices). In this manner, the lancet manufacturer can sell lancets of a standard design to users of various different types of lancing devices produced by one or more lancing device manufacturers, capturing a greater share of the market without the expense and difficulty of manufacturing multiple types of lancets to different specifications for each type of lancing device. Manufacturers of “standard” lancing devices (i.e., lancing devices accepting a lancet that is compatible with multiple types of existing lancing devices) may also prefer standardization, as it provides users of their lancing device with increased selection and availability of lancets.

As a result, manufacturers of lancets and lancing devices commonly adopt standard lancet profiles and cooperating standard lancet coupling profiles for lancing devices, conforming to one or more accepted industry norms. For example, many lancets are configured to have a generally cylindrical body profile having a diameter of about 6 mm, and many lancing devices have lancet coupling elements adapted to receive and engage lancets of this configuration.

In some instances, however, it is desirable to prevent the use of an unauthorized standard lancet in a lancet device. For example, some lancing devices include depth-control features that permit a user to selectively control the depth and/or size of a wound created by the lancet. These depth-control features typically require that one or more dimensions of the lancet be consistently maintained. For example, the distance that the lancet needle projects from the front face of the body of a lancet generally must be held constant in order for a depth control mechanism utilizing a stop for contacting the front face of the lancet body to function properly. Lancets having needle lengths shorter or longer than intended will result in wounds that are shallower or deeper, respectively, than desired. In addition, it has been found that some lancet manufacturers do not maintain the high level of quality control that is desired by manufacturers of higher-end lancing devices. If an end-user utilizes a generic lancet that is dull or otherwise defective, or that is unsterile, the user may incorrectly blame the manufacturer of the lancing device, rather than the manufacturer of the lancet, for increased pain, infection, or difficulties in use.

Accordingly, it has been found that needs exist for devices and methods that permit a manufacturer to have some measure of control over the types of lancets utilized with a lancing device. In addition, it has been found that needs exist for such devices and methods that make use of lancets that are compatible for use with standard lancing devices. It is to the provision of improved devices and methods meeting these and other needs that the present invention is primarily directed.

### SUMMARY OF THE INVENTION

Briefly described, according to a preferred form, the present invention includes a lancet that has a profile keyed to be accepted by a correspondingly keyed lancet coupling of a lancing device. In this manner, the lancing device is only usable with authorized lancets, and cannot accept unauthorized lancets that are not properly keyed. The lancet of the present invention, however, is compatible for use with standard unkeyed lancing devices, thereby increasing the potential market for the lancets. The present invention also includes a lancing device having a keyed lancet coupling with a profile that is adapted to accept only authorized lancets having a correspondingly keyed profile, but to reject unauthorized lancets that are not properly keyed, thereby allowing the manufacturer of the lancing device a measure of control over the quality and specifications of the lancets to be used with the lancing device.

In one aspect, the present invention is a lancet for use in combination with a lancing device having a keyed female coupling profile. The lancet preferably includes a keyed male coupling profile generally matching the keyed female coupling profile of the lancing device, and the keyed male coupling profile of the lancet is preferably circumscribable within a circle having a predetermined diameter.

In another aspect, the present invention is an improvement to a lancet compatible for engagement within a cylindrical lancet coupling of a standard lancing device, the improvement comprising a non-cylindrical coupling profile of the lancet.

In yet another aspect, the present invention is a lancet having a keyed coupling profile for engagement with a cooperating keyed coupling profile of a non-standard lancing device. The keyed coupling profile is preferably compatible for coupling with an unkeyed lancing device.

In still another aspect, the present invention is a method of preventing use of an unauthorized lancet in a specialized lancing device having a non-cylindrical coupling for engaging an authorized lancet, while permitting use of the authorized lancet in a standard lancing device having a cylindrical coupling. The method preferably includes providing the authorized lancet with a non-cylindrical coupling profile that engages the non-cylindrical coupling of the specialized lancing device and the cylindrical coupling of the standard lancing device.

These and other aspects, features and advantages of preferred forms of the present invention are described in greater detail herein with reference to example embodiments.

### BRIEF DESCRIPTION OF THE DRAWING FIGURES

FIGS. 1 a-e show a lancing device and lancet according to a first preferred form of the present invention.

FIGS. 2a-d show a lancet according to another preferred form of the present invention.

FIGS. 3a-d show a lancet according to another preferred form of the present invention.

FIGS. 4a-d show a lancet according to another preferred form of the present invention.

FIGS. 5a-d show a lancet according to another preferred form of the present invention.

FIGS. 6a-d show a lancet according to another preferred form of the present invention.

FIGS. 7a-d show a lancet according to another preferred form of the present invention.

FIGS. 8a-d show a lancet according to another preferred form of the present invention.

FIGS. 9a-d show a lancet according to another preferred form of the present invention.

FIGS. 10a-d show a lancet according to another preferred form of the present invention.

FIGS. 11a-d show a lancet according to another preferred form of the present invention.

FIGS. 12a-d show a lancet according to another preferred form of the present invention.

FIGS. 13a-d show a lancet according to another preferred form of the present invention.

FIGS. 14a-d show a lancet according to another preferred form of the present invention.

FIGS. 15a-d show a lancet according to another preferred form of the present invention.

FIGS. 16a-d show a lancet according to another preferred form of the present invention.

FIGS. 17a-f show a lancet according to another preferred form of the present invention.

FIGS. 18a-h show a lancet according to another preferred form of the present invention.

FIGS. 19a-d show a lancet according to another preferred form of the present invention.

### DETAILED DESCRIPTION OF PREFERRED EMBODIMENTS

Referring now to the drawing figures, in which like reference numbers refer to like parts throughout, preferred forms of the present invention will now be described by way of example embodiments. It is to be understood that the embodiments described and depicted herein are only selected examples of the many and various forms that the present invention may take, and that these examples are not intended to be exhaustive or limiting of the claimed invention.

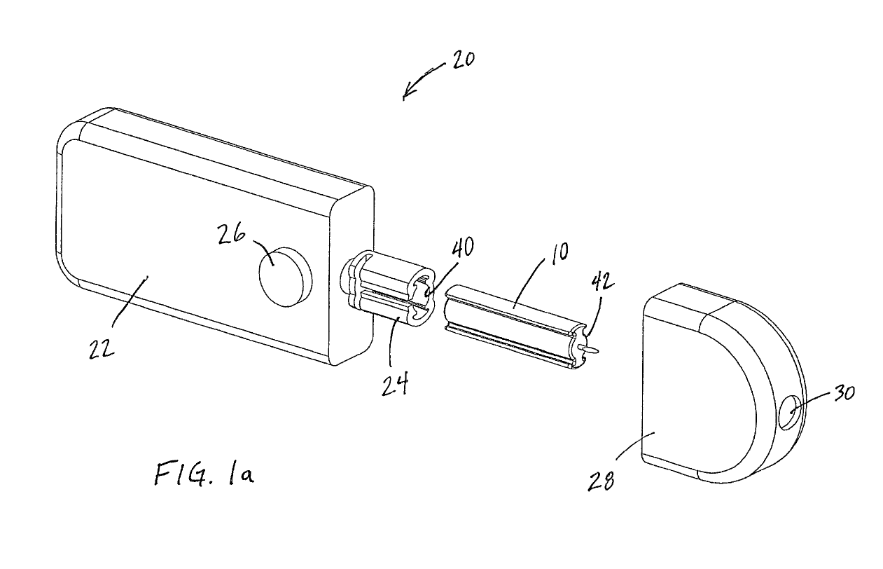

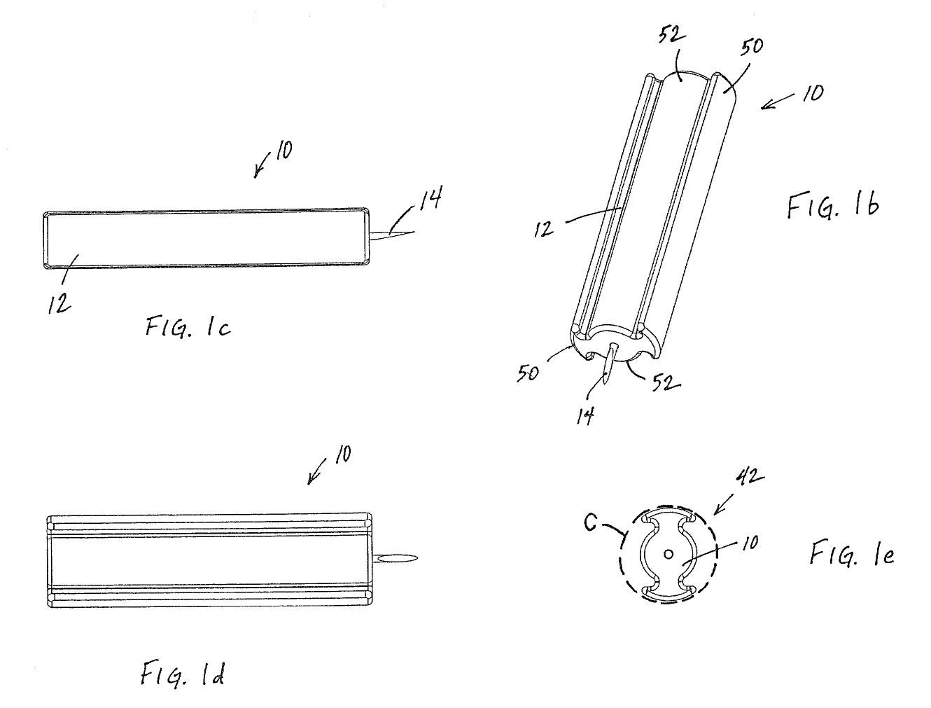

FIGS. 1a-e show a lancet 10 according to one preferred form of the present invention. The lancet 10 can be used alone by manually grasping the body 12 of the lancet and driving the point of the lancet needle 14 into the skin at a sampling site. More preferably, however, the lancet 10 is utilized in connection with a lancing device 20, as may be understood with reference to FIG. 1a. The lancing device 20 can be virtually any type of lancing device used with lancets. For example, the lancing device 20 can comprise a housing 22, with a lancet coupling 24, a triggering button 26, a drive mechanism (unshown) comprising a spring or other biasing means for driving the lancet from a retracted position to an extended position, a cocking mechanism (unshown) for arming the drive mechanism, and an endcap 28 for preventing unintended contact with the lancet 10 and/or for controlling the depth of penetration by the lancet. The endcap 28 typically comprises an opening 30 allowing passage of the needle 14 of the lancet in its extended position.

The lancet retainer or coupling 24 preferably comprises a first keyed coupling profile, and the lancet body 12 preferably comprises a second keyed coupling profile, the first and second keyed coupling profiles being adapted to cooperatively engage one another to securely but releasably retain the lancet 10 in the lancet coupling during use. For example, in the depicted embodiment, the lancet coupling 24 of the lancing device 20 comprises a female keyed coupling profile 40 and the body 12 of the lancet 10 preferably comprises a cooperating male keyed coupling profile 42. In preferred form, the male keyed coupling profile 42 is sized and shaped to generally match the female keyed coupling profile 40, and to be received therein with an interference or friction fit for insertion and removal by a user using moderate finger pressure. The retainer 24 optionally comprises resilient retaining and/or locking means, such as a split collar, one or more clips, interengaging projections and recesses, clamping elements, and/or the like, for holding and releasing the lancet 10.

The keyed coupling profile of the lancet 10 of the present invention preferably is compatible for use with the lancet coupling of a standard lancing device. In this manner, the keyed lancet 10 of the present invention can be used with the non-standard, keyed lancing device of the present invention, and can also be used in place of standard lancets in standard, unkeyed lancing devices. This allows a user to utilize keyed lancets according to the present invention in virtually any standard lancing device available, thereby increasing the potential market for such lancets.

Many standard lancing devices, for example, utilize a generally cylindrical socket type of lancet coupling for holding a standard lancet having a generally cylindrical outer body profile. The standard lancet typically has a predetermined body diameter, often for example, a diameter of about 6 mm, and the lancet coupling of a standard lancing device includes a cylindrical socket also having a diameter of about 6 mm. For compatibility with such a standard lancet coupling, the lancet 10 of the present invention preferably comprises a non-cylindrical keyed coupling profile that is capable of being received and engaged within an unkeyed cylindrical lancet coupling having a diameter of about 6 mm. Of course, it will be understood that the lancet and lancing device of the present invention can be sized for compatibility with standard, unkeyed lancet couplings of other sizes and geometries, such as for example lancet couplings having a diameter or other major dimension of less than 6 mm or of more than 6 mm, and/or a shape other than cylindrical (such as for example, a polygonal, oval, flat, curved or other cross-sectional shape).

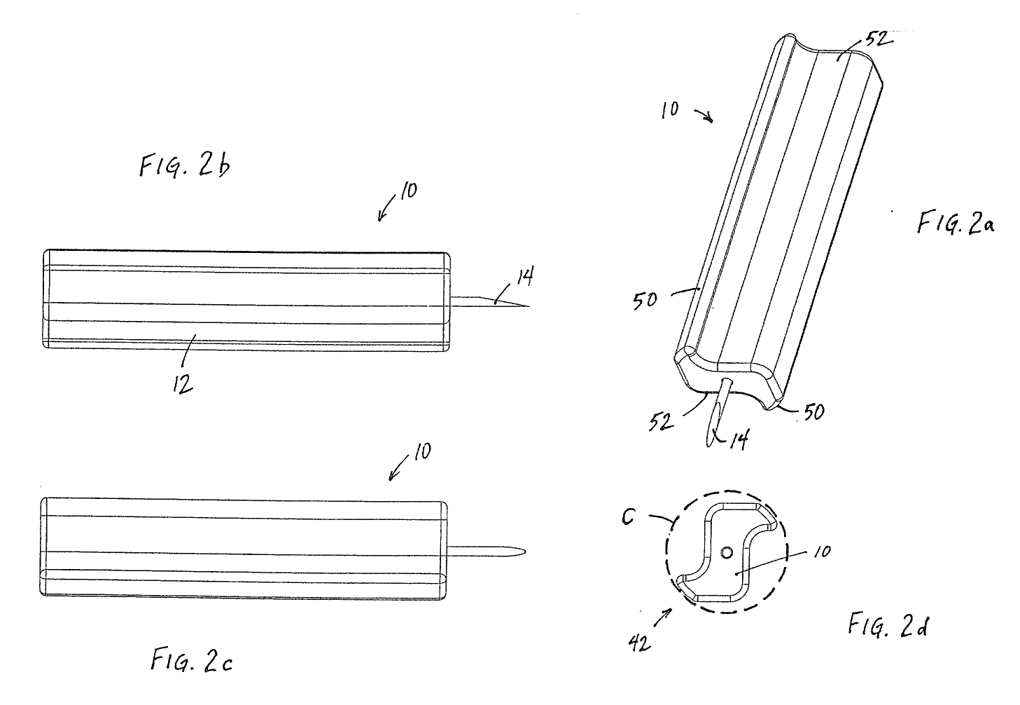

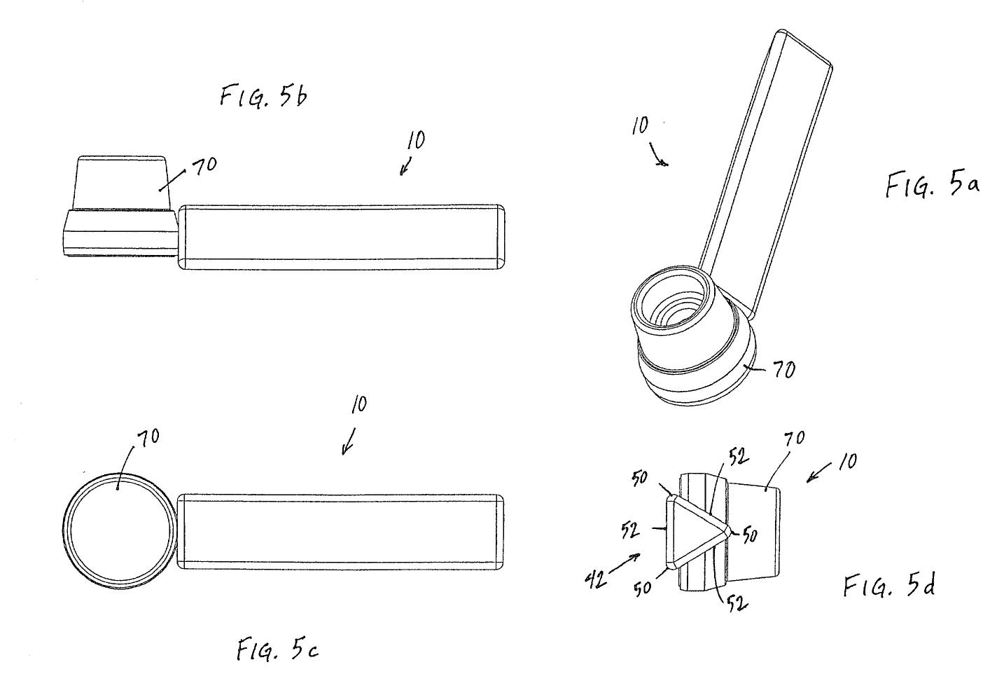

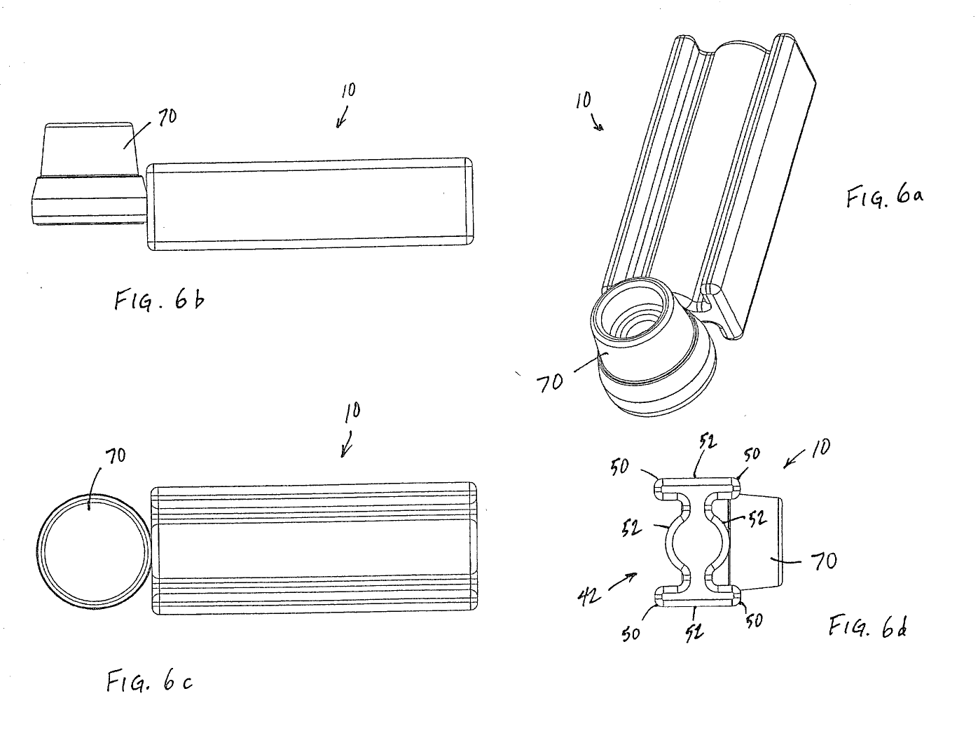

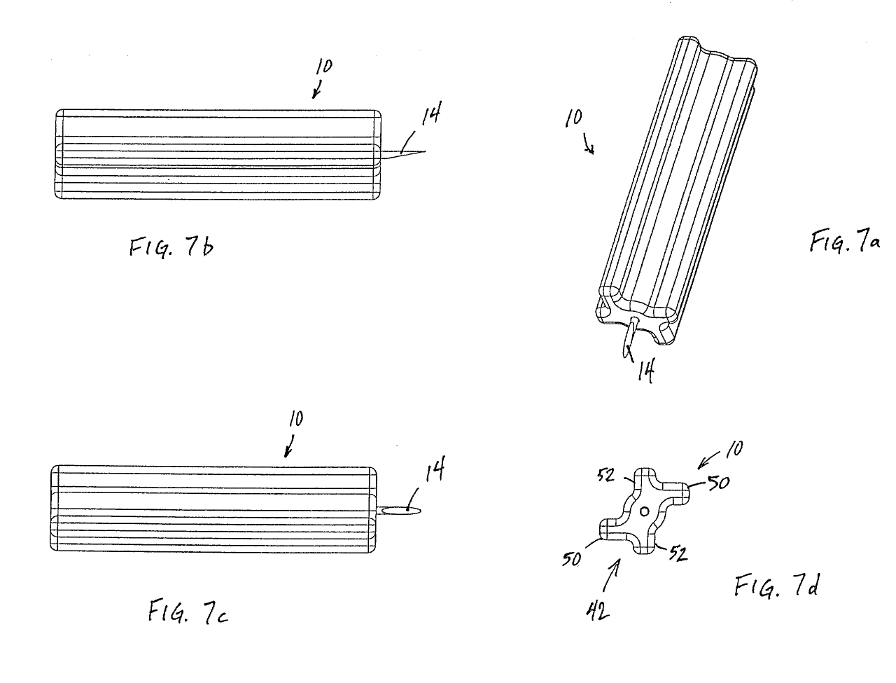

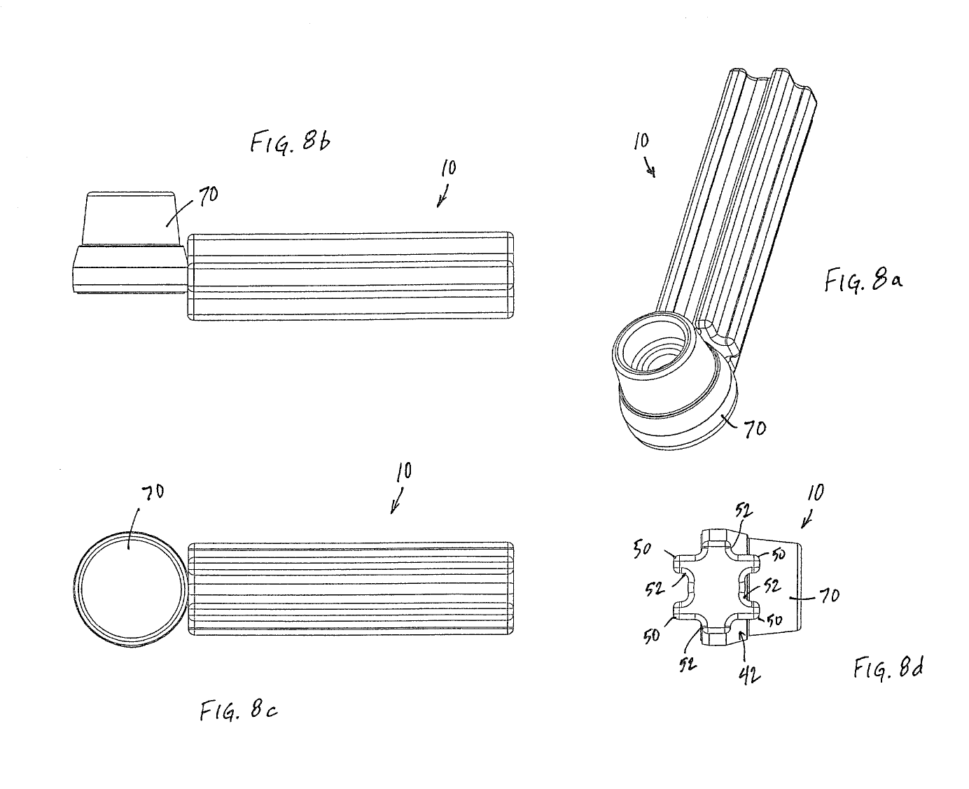

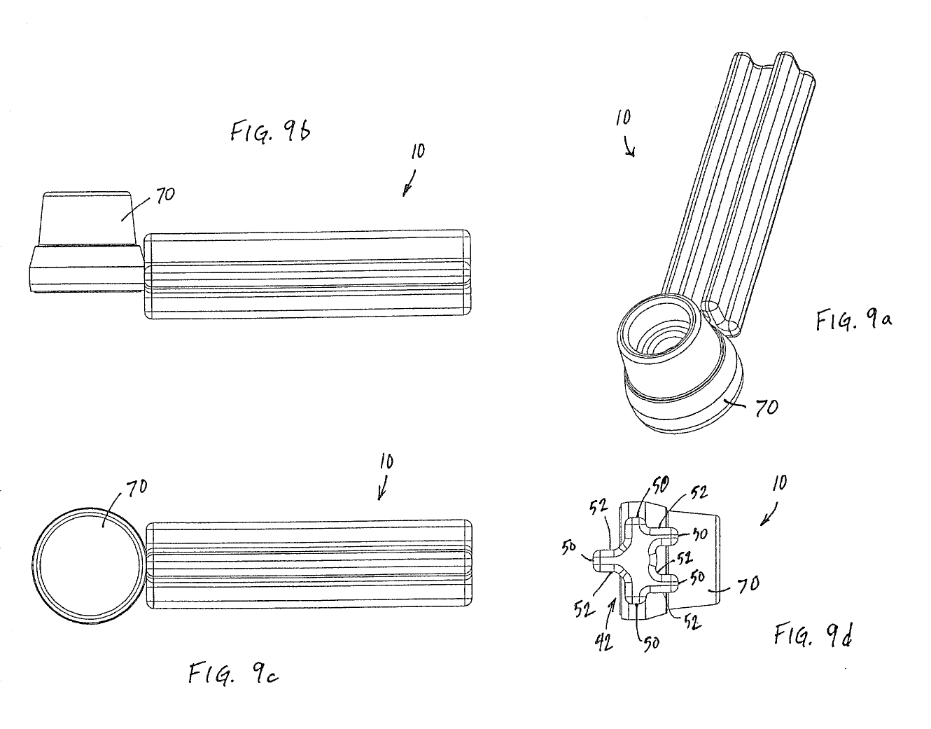

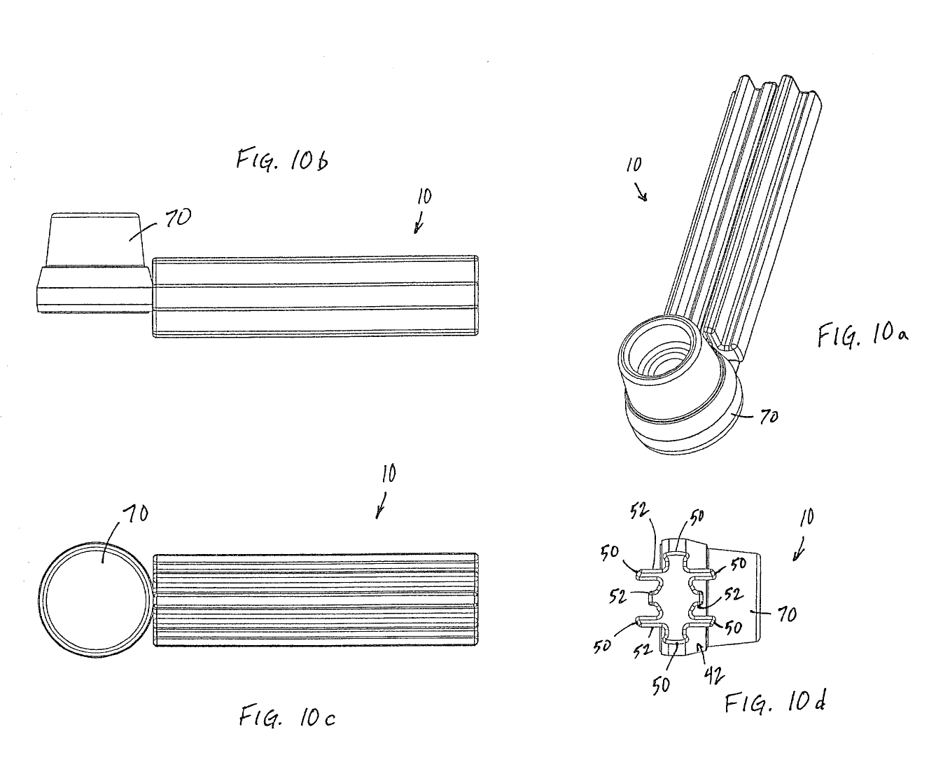

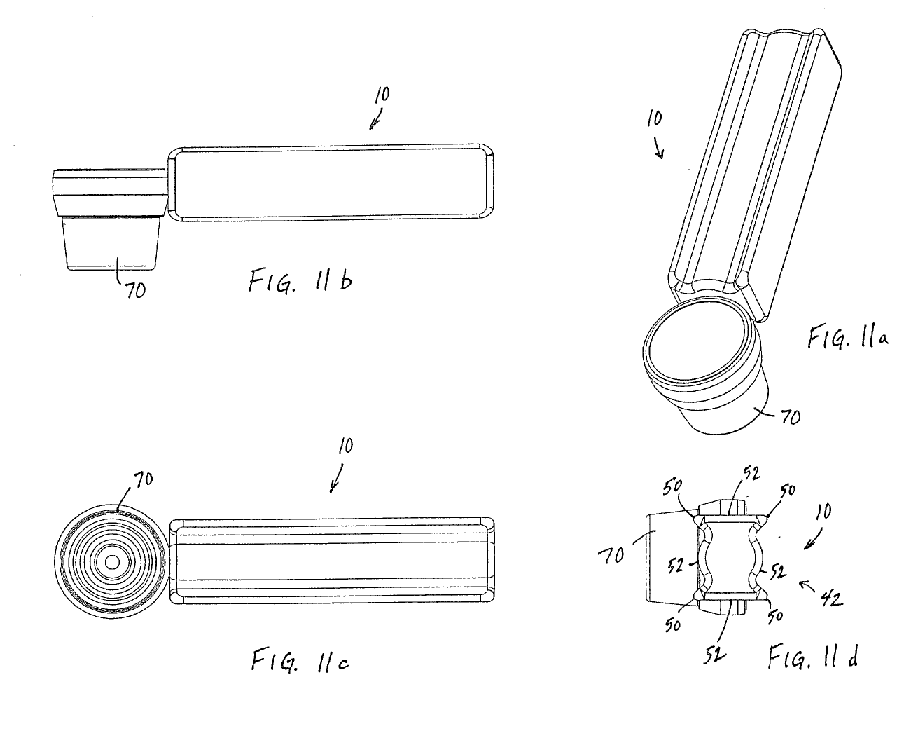

In some of its preferred forms, the keyed coupling profile of the lancet 10 of the present invention is circumscribable within a circle C having a predetermined diameter, such as for example a diameter of about 6 mm, corresponding to a dimension of a standard unkeyed lancet. FIGS. 1e and 2e show example keyed coupling profiles circumscribed within a circle C. Circle C preferably generally corresponds to the inner cylindrical geometry of the unkeyed lancet retainer of a standard lancing device. By providing at least two outer surface points of the keyed male coupling profile of the lancet 10 that maintain contact with an unkeyed lancet retainer, the lancet of the present invention is compatible with a standard unkeyed lancing device. Although the two points of contact may occur anywhere on the lancet 10, preferred forms of the present invention provide contact at points generally diametrically opposite one another on the circle C. More preferably, the lancet provides contact with three or more points on the circle C, as seen with reference to FIGS. 5d-15d. Also, although points of contact anywhere along the length of the lancet 10 may serve to retain the lancet in a lancet retainer of a lancing device, it is preferable to provide two or more lines of contact along substantially all of, or at least a portion of, the length of the lancet body 12.

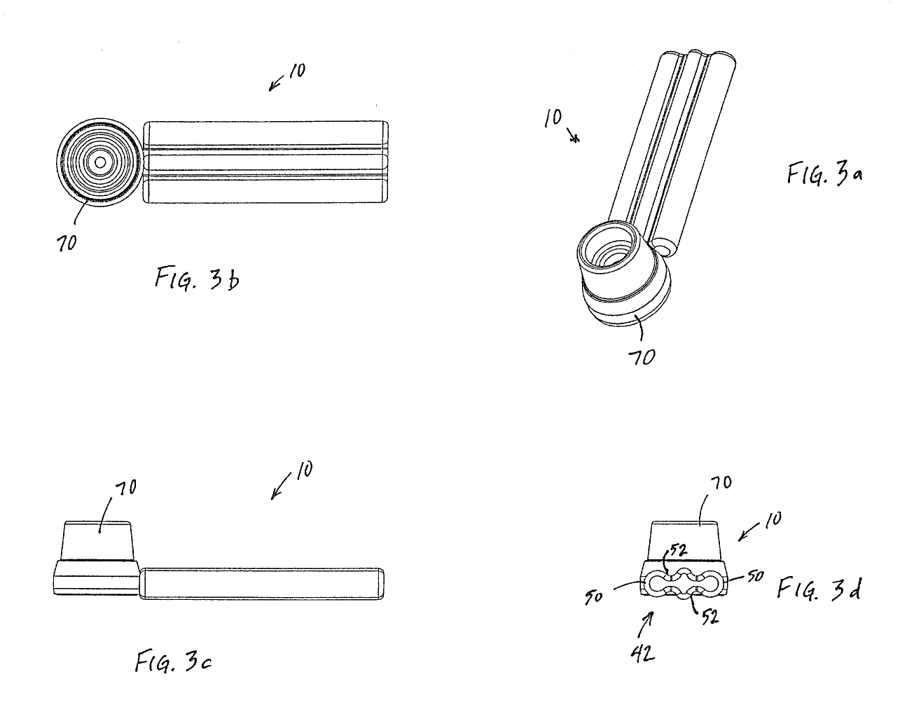

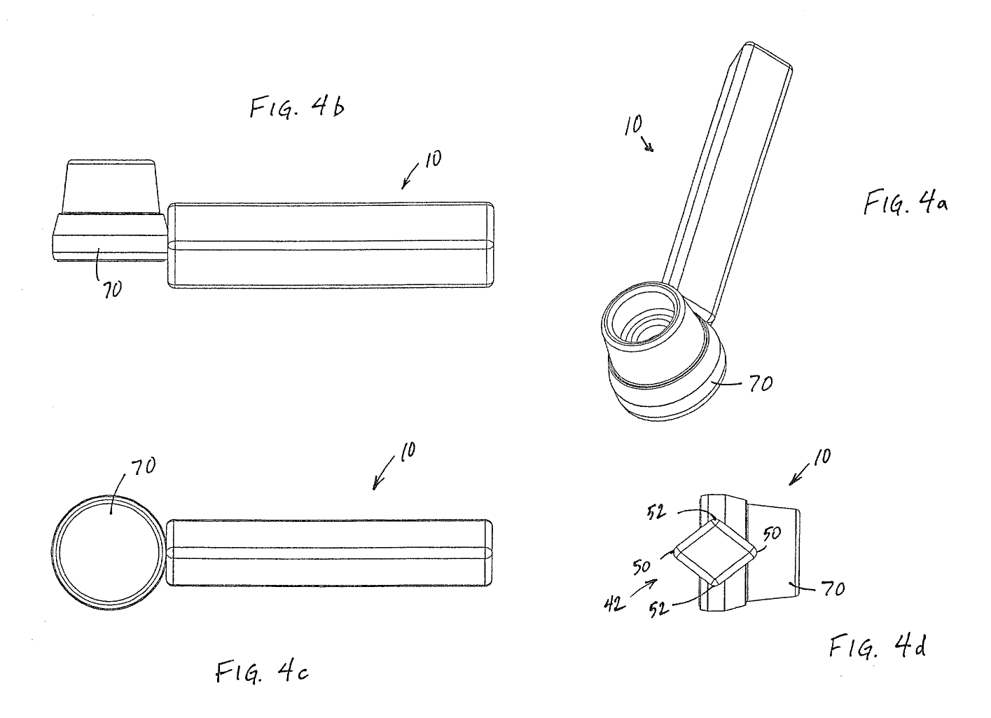

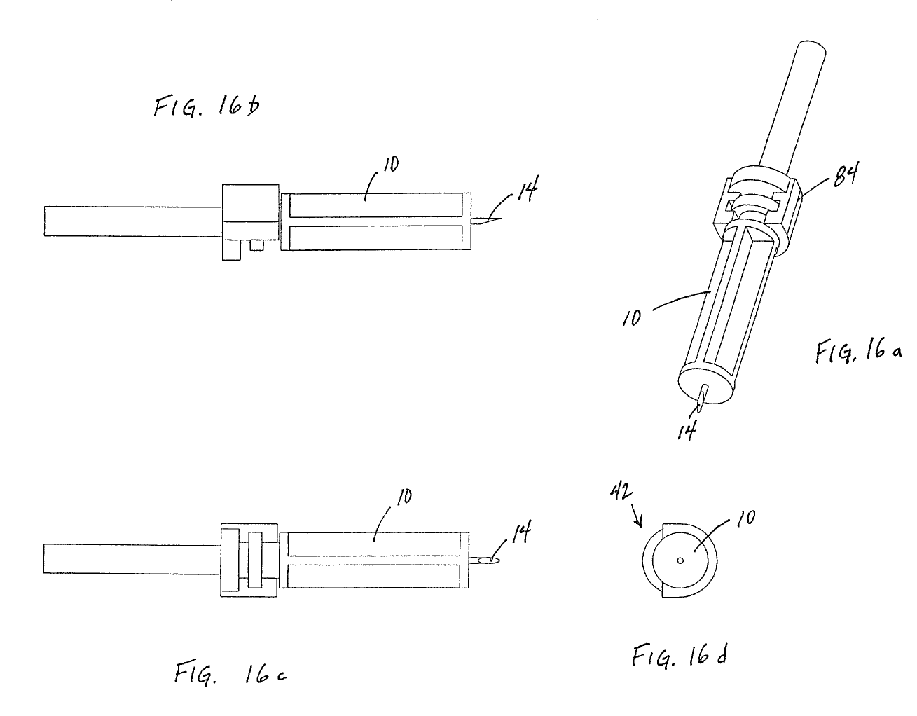

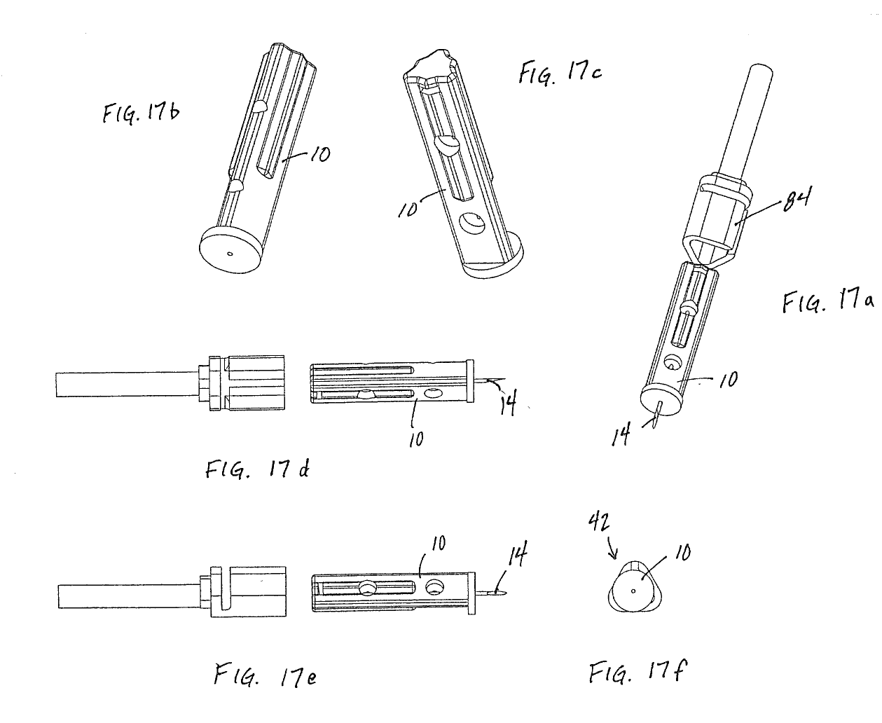

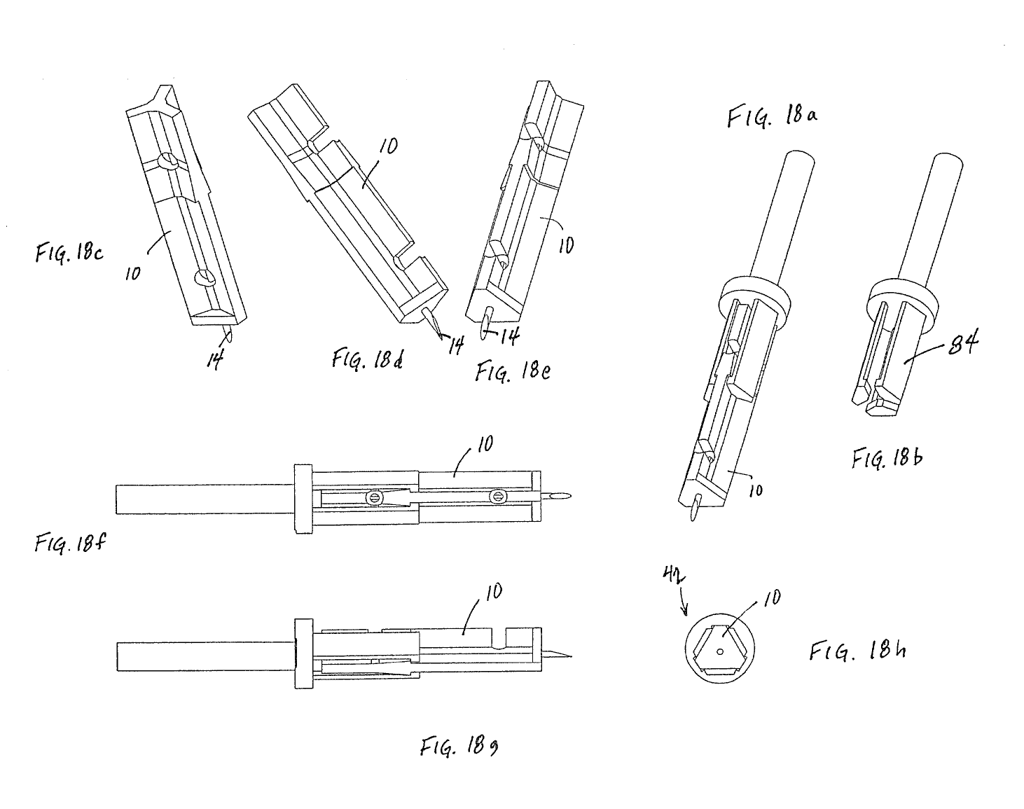

In further preferred forms, the keyed male coupling profile 42 of the lancet of the present invention comprises one or more outer surface portions 50 radiused to generally match the circumscribing circle C, and one or more inner surface portions 52 that are inwardly recessed from the outer surface portions. The coupling profile 42 of the lancet is non-cylindrical, having a non-circular cross-section at at least one portion along its length. The outer surface portions 50 are compatible for engagement of the lancet 10 within the lancet coupling of a standard lancing device. The inner surface portions 52, because they are recessed inwardly, do not interfere with engagement of the lancet 10 in the lancet coupling of a standard lancing device. As can be seen with reference to the example embodiments of FIGS. 1-19, a wide variety of lancet coupling profiles 42 can be utilized, including triangular, square, diamond, rectangular, polygonal, oval, S-shaped, I-shaped, X-shaped, hourglass shaped, single or multi-lobed, and various irregular shapes. Of course, it will be understood that the depicted embodiments are by way of example only, and are not limiting of the wide array of non-circular cross section coupling profiles 42 suitable for use in carrying out the present invention.

The lancing device 20 of the present invention preferably comprises a lancet coupling 24 having a coupling profile 40 sized and shaped to generally match and engage the coupling profile 42 of the lancet 10. The coupling profile 40 of the lancing device thus comprises one or more outer surface portions adapted to engage the outer surface portions 50 of the lancet, and one or more inner surface portions inwardly recessed therefrom for engaging the inner surface portions 52 of the lancet. The outer surface portions of the coupling profile 40 of the lancet coupling 24 of the lancing device 20 of the present invention preferably generally match the coupling profile of a lancet coupling of a standard lancing device, such as a generally cylindrical socket having a diameter of for example about 6 mm. Because the inner surface portions of the coupling profile 40 are inwardly recessed from the outer surface portions, the coupling profile 40 is non-cylindrical, having a non-circular cross-section at at least one portion along its length. As a result, the lancet coupling 24 of the lancing device 20 of the present invention does not accept a standard lancet such as a generally cylindrical lancet. If a user attempts to insert a standard lancet into the lancet coupling 24 of the lancing device 20 of the present invention, the inwardly projecting inner surface portions of the coupling profile 40 interfere and block passage of the unauthorized lancet into the lancet coupling 24. On the other hand, the inwardly recessed portions 52 of the keyed coupling profile 42 of a lancet 10 according to the present invention will accommodate the inwardly projecting inner surface portions of the coupling profile 40 and permit an authorized lancet to be received within the lancet coupling 24.

The lancet 10 of the present invention preferably comprises a removable protective cap or covering 70 over the needle 14 to preserve sterility of the needle. The cap 70 is preferably detachable from the body of the lancet by finger pressure to expose the needle when ready for use, and preferably further comprises a chamber for replacement over the needle 14 after use to prevent inadvertent needle sticks with a used lancet.

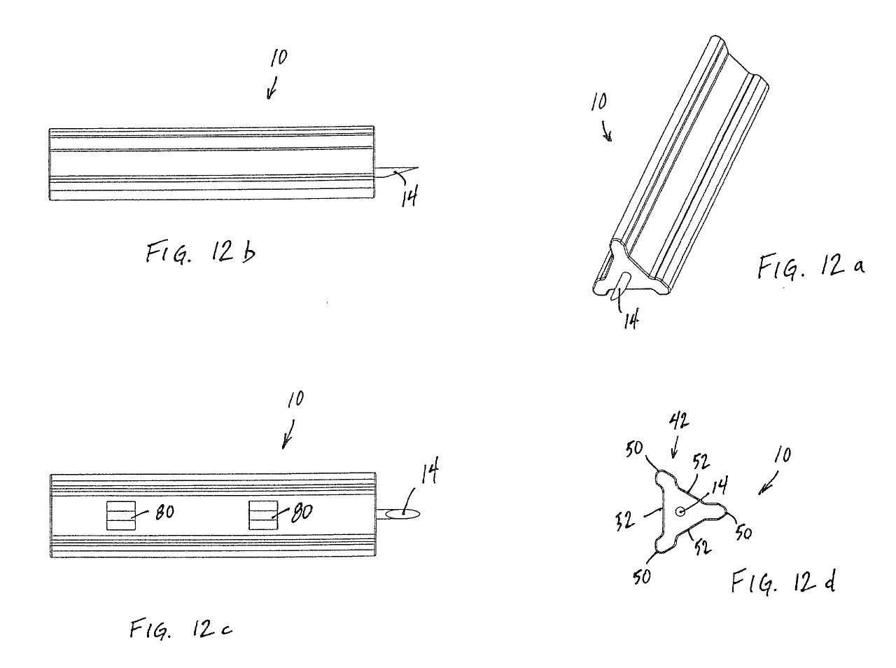

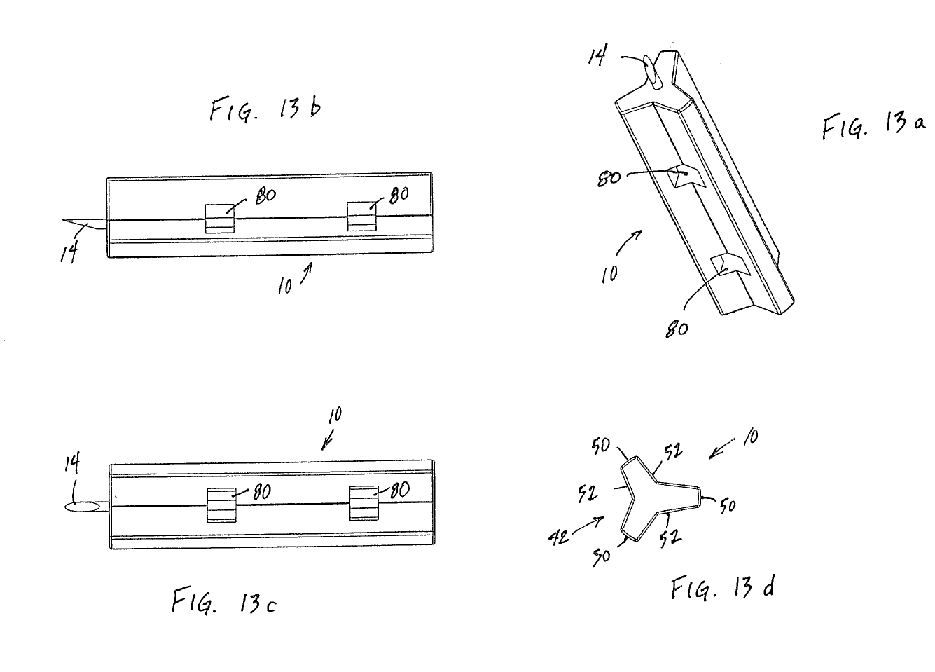

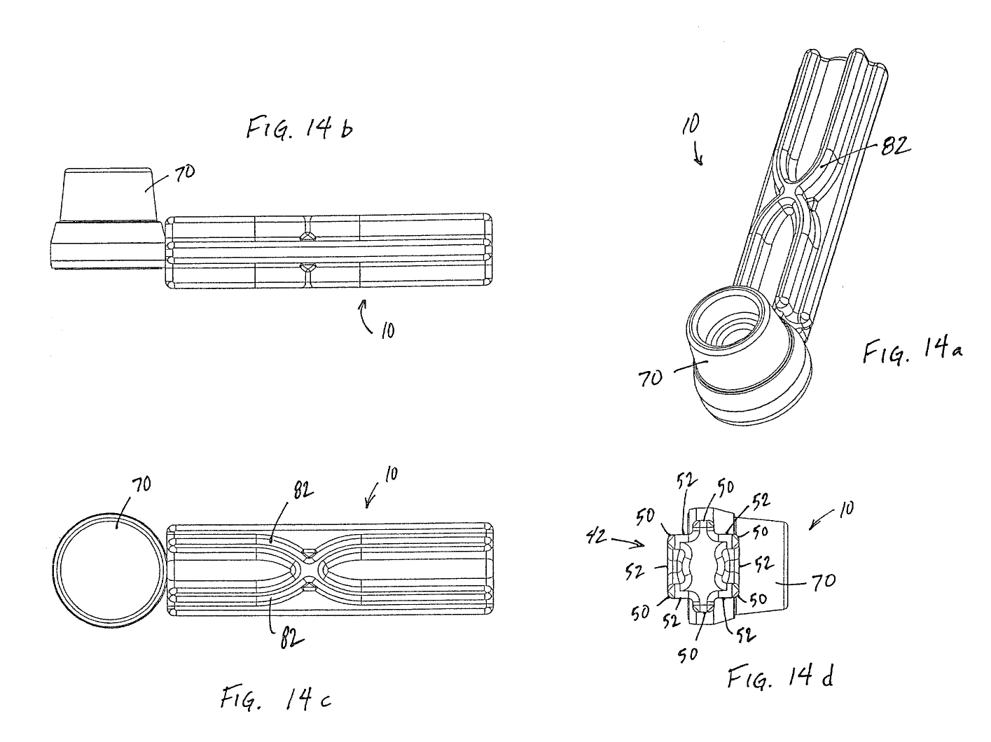

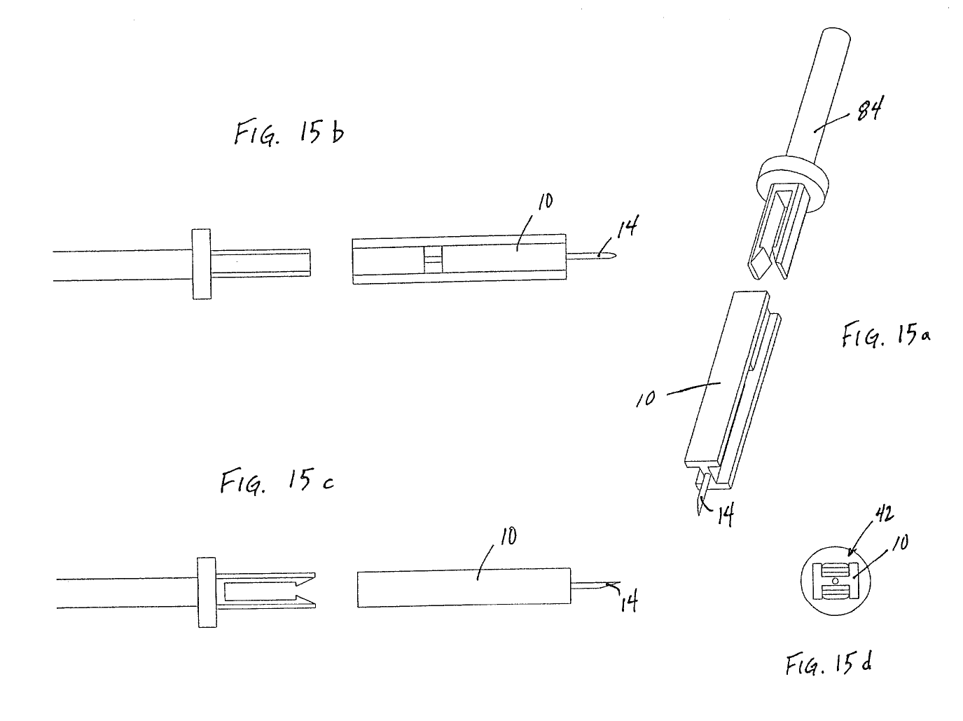

The lancet 10 optionally further comprises means for securing the lancet in a lancet coupling 24 of the lancing device 20. For example, and with reference to FIGS. 12-13, one or more recesses 80 can be provided in the lancet body 12 for receiving a cooperating projection (unshown) of a lancet coupling 24 of a lancing device. Alternatively, as shown in FIG. 14, the lancet 10 can comprise one or more channels, projections or ribs 82 capable of engagement with cooperating features of the lancet coupling 24 of a lancing device. In other alternate embodiments of the invention, for example as shown in FIGS. 15-19, a lancet clip 84 is provided for securing the lancet 10 to the lancet coupling 24. The lancet clip 84 preferably comprises one or more resilient fingers, recesses or projections for engaging cooperating surface features of the lancet 10. In other embodiments, the lancet 10 is secured to the lancet coupling 24 magnetically, by friction, by one or more fasteners, or the like.

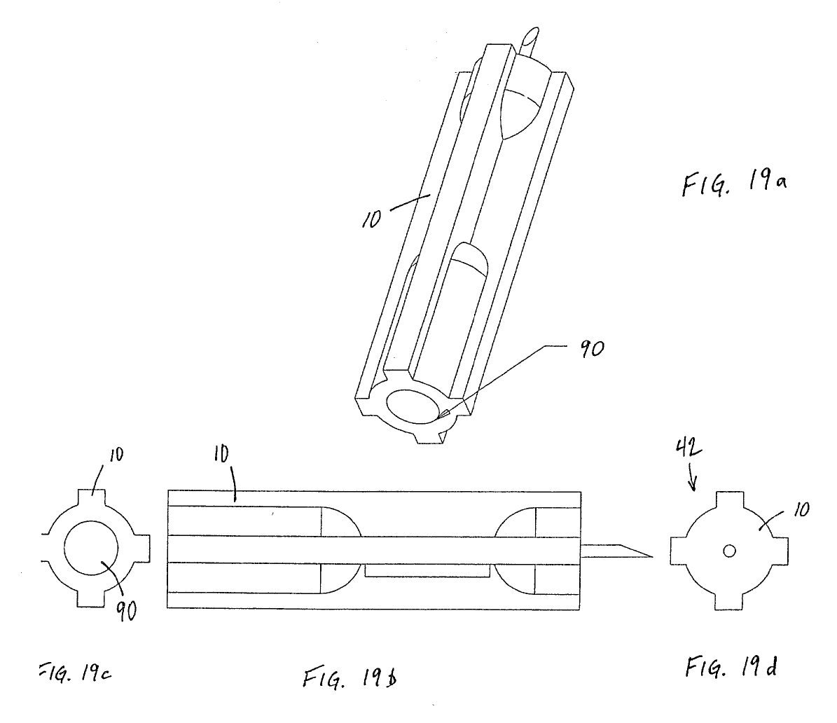

The non-cylindrical coupling profile of the lancet 40 can also comprise internal recesses or inner surface features. For example, as shown in FIG. 19, the lancet 10 comprises an internal recess or bore 90. In this embodiment of the invention, the coupling profile 42 of the lancet coupling 24 of the lancing device 20 preferably comprises a projection or pin (unshown) sized and shaped to be received within the internal recess or bore 90 to permit the lancet to be inserted in the lancet coupling. Although a circular bore 90 is depicted, the internal recess or bore can take various shapes, and the projection or pin of the lancet coupling 24 will be keyed to cooperate therewith. A standard lancet lacking an appropriately keyed internal recess or bore 90 will be blocked by the projection or pin from insertion into the lancet coupling 24.

The present invention further comprises the combination of a lancet 10, substantially as described, with a lancing device 20, substantially as described. The present invention also comprises a method of preventing use of an unauthorized lancet in a lancing device, substantially as described, but still permitting use of the authorized lancet 10, substantially as described, in a standard lancing device. An authorized lancet is provided with a non-cylindrical coupling profile 42 that engages a non-cylindrical coupling 24 of lancing device 20, and yet is still compatible with the cylindrical coupling of a standard lancing device. On the other hand, the cylindrical profile of a standard lancet is not accepted into the non-cylindrical coupling 24 of the lancing device 20.

The above description and appended drawings are representative of example embodiments of the present invention. The full spirit and scope of the invention, however, is not limited to any particular embodiment or embodiments. Thus, it will be readily apparent to those of ordinary skill in the art that many additions, modifications and deletions can be made to the described embodiments without departing from the spirit and scope of the invention.

What is claimed is:

1. A lancet for use with a lancing device having a keyed female coupling profile, said lancet comprising a keyed male coupling profile generally matching the keyed female coupling profile of the lancing device, said keyed male coupling profile of said lancet circumscribable within a circle having a predetermined diameter.

2. The lancet of claim 1, wherein said keyed male coupling profile of said lancet comprises at least one outer surface portion radiused to match the circumscribing circle, and at least one inner surface portion that is inwardly recessed from said outer surface portion.

3. The lancet of claim 1, wherein said keyed male coupling profile of said lancet comprises at least two outer surface points contacting the circumscribing circle.

4. The lancet of claim 1, wherein said keyed male coupling profile of said lancet has a maximum outer dimension of about 6 mm.

5. The lancet of claim 1, wherein said keyed male coupling profile of said lancet is non-cylindrical.

6. The lancet of claim 1, wherein said keyed male coupling profile of said lancet has a polygonal cross section.

7. The lancet of claim 1, wherein said keyed male coupling profile of said lancet comprises an internal recess.

8. The lancet of claim 7, wherein said keyed male coupling profile of said lancet comprises a generally cylindrical outer surface profile.

9. In a lancet compatible for engagement within a cylindrical lancet coupling of a standard lancing device, the improvement therein comprising a non-cylindrical coupling profile of said lancet.

10. The lancet of claim 9, wherein said non-cylindrical coupling profile is circumscribable within a circle and comprises at least two outer surface points lying on the circumscribing circle.

11. The lancet of claim 9, wherein said non-cylindrical coupling profile of said lancet has a maximum outer dimension of about 6 mm.

12. The lancet of claim 9, wherein said non-cylindrical coupling profile of said lancet has a polygonal cross section.

13. The lancet of claim 9, wherein said non-cylindrical coupling profile of said lancet comprises an internal recess.

14. A lancing device having a first keyed coupling profile for engaging a lancet, said first keyed coupling profile comprising a non-circular cross section at at least one point along a length thereof.

15. The lancing device of claim 14, wherein said first keyed coupling profile comprises a female coupling profile.

16. The lancing device of claim 14, wherein said first keyed coupling profile is circumscribable within a circle and comprises at least two outer surface points lying on the circumscribing circle.

17. The lancet of claim 16, wherein said first keyed coupling profile has a maximum outer dimension of about 6 mm.

18. In combination:
a lancing device having a first keyed coupling profile comprising a non-circular cross section at at least one point along a length thereof; and
a lancet having a second keyed coupling profile for cooperative engagement within the first keyed coupling profile of said lancet.

19. The combination of claim 18, wherein the second keyed coupling profile of said lancet comprises at least one outer surface portion and at least one inner surface portion inwardly recessed from said outer surface portion.

20. A method of preventing use of an unauthorized lancet in a non-standard lancing device having a non-cylindrical lancet coupling for engaging an authorized lancet, while permitting use of the authorized lancet in a lancet coupling of a standard lancing device, said method comprising providing the authorized lancet with a non-cylindrical coupling profile that is compatible with the non-cylindrical lancet coupling of the non-standard lancing device and the lancet coupling of the standard lancing device.
